(19) United States
(12) Patent Application Publication
(10) Pub. No.: US 2023/0380425 A1
(43) Pub. Date: Nov. 30, 2023

(54) COLORLESS ANTIMICROBIAL COMPOSITION

(71) Applicant: Argaman Technologies Ltd., Migdal Haemek (IL)

(72) Inventor: Jeffrey S. Gabbay, Jerusalem (IL)

(21) Appl. No.: 18/249,596

(22) PCT Filed: Oct. 19, 2021
(86) PCT No.: PCT/IL2021/051241

§ 371 Date: Apr. 19, 2023

Related U.S. Application Data
(60) Provisional application No. 63/094,350, filed on Oct. 21, 2020.

(51) Int. Cl. A01N 59/20; A01N 25/34; A01N 25/10; C08J 3/22; D06M 11/42; D06M 11/46; D06M 11/71; D01F 1/10

(52) U.S. Cl. CPC A01N 59/20; A01N 25/34; A01N 25/10; C08J 3/226; D06M 11/42; D06M 11/46; D06M 11/71; D01F 1/103; C08J 2423/06; C08J 2423/12; C08J 2455/02; C08J 2477/10

(57) ABSTRACT

A composition with antimicrobial properties for impregnation of staple fibers and filaments comprising: titanium dioxide; a salt comprising silver and phosphate ions; copper oxide; and a mixed oxidation state silver oxide. A masterbatch formulation which includes the composition defined above is also taught as is a method for producing polymer filaments using the masterbatches. A material having wound healing properties and a material having beneficial cosmetic properties are taught, the materials comprising a polymer having incorporated therein the composition described above.

### FIELD OF THE INVENTION

The present invention relates to a colorless antimicrobial composition comprising a combination of multiple metal oxide and inorganic salt compounds which can be used in compounding a masterbatch formulation and in impregnating natural and synthetic fibers.

### BACKGROUND OF THE INVENTION

Today more and more microbes have developed partial or complete immunity to antibiotics developed in the twentieth century. Alternative methods for controlling them are being looked at. Surprisingly, various ancient methods of microbe control that have fallen into disuse are now being revisited and reviewed for efficacy. One of these methods uses the antimicrobial effect of various metal ions. The mechanism of their operation is believed to be significantly different from the antibiotics and other organic materials that have been used to control microbes for the last 100 years. The metal ions typically used and studied are copper and silver.

U.S. Pat. No. 7,169,402 encompasses antimicrobial and antiviral polymeric material having microscopic particles of ionic copper encapsulated therein and protruding from surfaces thereof.

US Patent Application Publication No. 2008/0193496 discloses polymeric master batch for preparing an antimicrobial and antifungal and antiviral polymeric material comprising a slurry of thermoplastic resin, an antimicrobial and antifungal and antiviral agent consisting essentially of water insoluble particles of ionic copper oxide, a polymeric wax and an agent for occupying the charge of said ionic copper oxide.

U.S. Pat. No. 6,436,420 is related to fibrous textile articles possessing enhanced antimicrobial properties prepared by the deposition or interstitial precipitation of tetrasilver tetroxide (Ag₄O₄) crystals within the interstices of fibers, yarns and/or fabrics forming such articles.

US Patent Application Publication No. 2018/0020670 is directed to materials having antimicrobial properties, which include a polymer having incorporated therein a synergistic combination of at least two metal oxide powders, including a mixed oxidation state oxide of a first metal and a single oxidation state oxide of a second metal.

One of the major disadvantages of using copper and silver-containing compositions in textile industry is natural dyeing of the fabric to which the composition is applied [Eremenko, A M et al, “Antibacterial and Antimycotic Activity of Cotton Fabrics. Impregnated with Silver and Binary Silver/Copper Nanoparticles.” Nanoscale research letters vol. 11,1 (2016): 28]. The use of such compositions is therefore generally limited to dark-colored textiles in which the brownish color of copper or silver oxide is less noticeable. While yarn or fabric containing copper or silver oxides may be bleached and/or optically whitened, this adds a further processing step and increases the overall production cost.

There exists an unmet need for a highly efficient antimicrobial material based on copper or silver, which would not affect color of the fibers, yarns or fabrics to which it is applied.

### SUMMARY OF THE INVENTION

The present invention relates to antimicrobial compositions, methods for the production of these compositions, and use of these compositions for a variety of applications, such as, but not limited to, controlling the proliferation of microbes, controlling negative odors caused by microbes, stimulation of cell proliferation which assists in the closure of wounds and the improvement of skin elasticity and skin texture.

The compositions of the present invention advantageously provide varying release kinetics for the active ions in the compositions due, at least partly, to the different oxidation potentials of the metal compounds in the compositions. Without being limited to a specific theory, these various compounds are thought to act as a stimulant for ionic release upon one another.

More particularly, the invention relates to the inclusion of these compositions into polymers and for attachment to natural fibers or as a coating thereto. As a result, the properties of the compositions are introduced into the products made from these materials without changing any of the physical characteristics of the polymers or cellulose-based fibers. More particularly, the compositions of the present invention are white in color, despite the presence of copper oxide in the mixtures and thereby they do not affect the color of the fibers, yarns or fabrics to which they are applied. Furthermore, the invention can be thought of as relating to the use of multiple metal salts or metal oxide catalysts which result in a chemical formulation that is far more potent than any single metal or metal oxide or any known combination thereof.

The present invention is based in part on a surprising finding that the addition of a whitening agent to an antimicrobial composition containing copper oxide and tetrasilver tetroxide, which provided an essentially white composition, significantly reduced the efficiency of said composition. In order to mask the natural hue of copper oxide in the mixture without inhibiting its antimicrobial activity, inclusion of an additional components into the mixture was required—said compounds being an inorganic salt comprising silver phosphate and a mixed oxidation state silver oxide.

It is an object of the present invention to provide a colorless multi-component composition with antimicrobial capabilities greater than any one of its single constituents.

It is another object of the present invention to rid the resultant fibers, yarns, and fabrics of any residual copper color, inter alia thereby providing for more uniform color during subsequent dyeing processes.

It is a further object of the invention to provide a wound healing composition that will provide for better results than previous inorganic wound healing compositions.

It is yet a further object of the invention to provide a material for treating cosmetic problems, such as increasing skin elasticity, skin texture, and skin hydration, and reducing crow's feet, wrinkles, and mottled hyper-pigmentation.

In one aspect, the present invention provides a colorless composition with antimicrobial properties for impregnation of filaments, sliver fibers and staple fibers, the composition comprising the following components: titanium dioxide (TiO₂); a salt comprising silver and phosphate ions; copper oxide; and a mixed oxidation state silver oxide.

Copper oxide can be selected from the group consisting of cuprous oxide, cupric oxide and mixtures thereof. In certain embodiments, copper oxide is cuprous oxide.

The salt comprising silver and phosphate ions can be selected from the group consisting of silver phosphate (Ag₃PO₄), silver sodium hydrogen zirconium phosphate (Ag_{(0.1-0.5)}Na_{(0.1-0.8)}H_{(0.1-0.8)}Zr₂(PO₄)₃), and mixtures thereof. In some embodiments, silver sodium hydrogen zirconium phosphate is selected from the group consisting of Ag_{0.18}Na_{0.57}H_{0.25}Zr₂(PO₄)₃, Ag_{0.46} Na_{0.29}H_{0.25}Zr₂(PO₄)₃, and mixtures thereof.

In some embodiments Ag₃PO₄ is encapsulated by a glass, zirconium or zeolite encapsulant.

The mixed oxidation state silver oxide can be selected from the group consisting of Ag₄O₄, Ag₂O₂, and mixtures thereof.

In some embodiments, the composition further comprises at least one of a zinc species and elemental silver (Ag). The zinc species can be selected from the group consisting of elemental zinc, ZnO, and mixtures thereof.

In certain embodiments, the composition comprises the following components in the following weight percentages out of the total weight of the composition: about 70-85% (w/w) TiO₂; about 10-25% (w/w) of the salt comprising silver and phosphate ions; about 0.2-10% (w/w) copper oxide; and about 0.01-1.5% (w/w) of the mixed oxidation state silver oxide.

According to some embodiments, the composition further comprises about 1.5-5% (w/w) Zn species out of the total weight of the composition.

According to some embodiments, the composition further comprises about 0.05-0.5% (w/w) elemental Ag out of the total weight of the composition.

In some embodiments, particulates of the components of the composition have a diameter with a D50 ranging from about 100 nm to about 10 μm.

In some embodiments, particulates of the components of the composition have a diameter with a D50 ranging from about 100 nm to about 5 μm.

In another aspect, there is provided a masterbatch formulation which comprises the composition according to the various embodiments presented hereinabove and a carrier polymer.

In some embodiments, the carrier polymer is present in the masterbatch formulation in a weight percent of about 60-99% out of the total weight of the masterbatch formulation. The carrier polymer can be selected from the group consisting of polyethylene, polypropylene, polybutylene terephthalate (PBT), polyolefins, acrylonitrile-butadiene-styrene (ABS), polyaramids, and mixtures thereof.

In some embodiments, the masterbatch formulation further comprises a wax for encapsulating the components of the composition. In further embodiments, the wax is present in the masterbatch formulation in a weight percent of about 0.1-1.0% out of the total weight of the masterbatch formulation. The wax can be selected from the group consisting of polyethylene terephthalate (PET), polyester, polyalkene waxes and mixtures thereof.

In some embodiments, the masterbatch formulation further comprises a dispersing polymer for dispersing the components of the formulation in the carrier polymer. In further embodiments, the dispersing polymer is present in the masterbatch formulation in a weight percent of about of 0.1-1.0% out of the total weight of the masterbatch formulation. The dispersing polymer can be selected from the group consisting of polymethylmethacrylate (PMMA) and silica.

In another aspect, there is provided a method for producing antimicrobial polymer filaments comprising the steps of: (a) providing and melting a substrate polymer by passing it through a heated extruder; (b) adding the masterbatch formulation according to the various embodiments presented hereabove, to the melted substrate polymer; and (c) extruding a filament containing the masterbatch formulation uniformly dispersed therein, wherein the masterbatch formulation constitutes about 1-10% (w/w) of the substrate polymer.

According to some embodiments, the method further comprises a step of cutting the filament into staple fibers.

According to some embodiments, the substrate polymer is selected from the group consisting of polyethylene, polypropylene, polybutylene terephthalate, polyolefins, ABS, polyaramids, and mixtures thereof.

In another aspect there is provided a method for producing antimicrobial polymer filaments comprising the steps of: (a) providing and melting a substrate polymer by passing it through a heated extruder, the extruder extruding substrate polymer filaments; and (b) sprinkling the colorless composition with antimicrobial properties according to the various embodiments presented hereinabove, on an external surface of the substrate polymer filaments after they emerge from the extruder, thereby imparting antimicrobial properties to the filaments.

In another aspect there is provided a method for producing antimicrobial natural sliver fibers comprising the steps of: (a) providing at least one ribbon of sliver fibers; (b) dispensing a paste comprising the colorless composition with antimicrobial properties according to the various embodiments presented hereinabove, water and a thickening agent, on the at least one sliver fiber ribbon; and (c) conveying the paste-coated at least one sliver fiber ribbon through a sonotrode.

In another aspect, there is provided a material comprising filaments, sliver fibers or staple fibers having incorporated therein the colorless composition with antimicrobial properties according to the various embodiments presented hereinabove.

In some embodiments, the components of the composition are dispersed substantially uniformly throughout the bulk of the filaments, sliver fibers or staple fibers.

In some embodiments, at least 0.25% of the total weight of the components of the composition are present on the surface of the filaments, sliver fibers or staple fibers.

The filaments, sliver fibers or staple fibers of the material can be formed into a yarn, a fabric, or a finished textile product.

In some embodiments, said filaments or staple fibers are made from a polymer. The polymer can be selected from the group consisting of polyamide, polyester, polyalkene, polysiloxane, nitrile, polyvinyl acetate, starch-based polymer, cellulose, cellulose-based polymer, and mixtures thereof. In some embodiments, the composition is encapsulated in a wax before being incorporated into the polymer.

The wax can be selected from the group consisting of PET, polyester, polyalkene waxes, and mixtures thereof.

In some embodiments, said filaments, sliver fibers or staple fibers are made from a natural material. The natural material can be selected from cotton, silk, wool, and mixtures thereof.

In some embodiments, the material is for use in combating or inhibiting the activity of microbes or micro-organisms, selected from the group consisting of gram-positive bacteria, gram-negative bacteria, fungi, parasites, mold, spores, yeasts, protozoa, algae, acarii and viruses.

In some embodiments, the material is for use in skin regeneration processes, selected from the group consisting of wound healing, accelerated wound closure, and wound healing with reduced scarring.

In some embodiments, the material is for use in a cosmetic treatment, selected from the group consisting of reducing wrinkles, reducing crows-feet, reducing skin hyper-pigmentation, reducing facial and neck lines, reducing erythema, reducing edema, softening of skin and improving skin elasticity, wherein the filaments, sliver fibers or staple fibers are in direct contact with part of a user's face or neck requiring said cosmetic treatment. In some related embodiments, the components of the composition are in contact with a fluid.

According to some embodiments, the filaments, sliver fibers or staple fibers are used to produce facial masks, eye masks, scarves, clothing items, bedding textiles, medical textiles, bandages or sutures.

In another aspect, there is provided a colorless composition with antimicrobial properties for impregnation of filaments, sliver fibers and staple fibers, the composition being prepared by mixing the following components: TiO₂; a salt comprising silver and phosphate ions; copper oxide; and a mixed oxidation state silver oxide.

According to some embodiments, the composition is prepared by mixing the following components in the following weight percentages out of the total weight of the composition: about 70-85% (w/w) TiO₂; about 10-25% (w/w) of the salt comprising silver and phosphate ions; about 0.2-10% (w/w) copper oxide; and about 0.01-1.5% (w/w) of the mixed oxidation state silver oxide.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION

Before explaining several embodiments of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

It should be noted that throughout this document all data is exemplary. It is used solely to present and explain the invention and as a possible implementation of the invention and it is not intended to limit the invention. Similarly, the invention has been described in relation to particular embodiments which are intended in all respects to be illustrative rather than restrictive.

As used herein “comprising” or “comprises” or variants thereof is to be interpreted as specifying the presence of the stated features, integers, steps, or components as referred to, but does not preclude the presence or addition of one or more additional features, integers, steps, components or groups thereof. Thus, for example, a method comprising given steps may contain additional steps.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a larger range of numerical values is recited herein unless otherwise stated, the range is intended to include the endpoints thereof, and all values within the range. It is also intended to include all ranges within the upper and lower values of the endpoints of the specified range. It is not intended that the scope of the invention be limited to the specific values recited when defining a range.

As used herein and in the appended claims the singular forms “a”, “an,” and “the” include plural references unless the content clearly dictates otherwise. Thus, for example, reference to “a fiber” includes a plurality of such fibers and equivalents thereof known to those skilled in the art, and so forth. It should be noted that the term “and” or the term “or” is generally employed in its sense including “and/or” unless the content clearly dictates otherwise.

As used herein, the term “about”, when referring to a measurable value such as an amount, a temporal duration, and the like, is meant to encompass variations of +/−10%, more preferably +/−5%, even more preferably +/−1%, and still more preferably +/−0.1% from the specified value, as such variations are appropriate to perform the disclosed methods.

The present invention discussed herein provides the following new features:

1. A composition for use as an antimicrobial agent with textiles, the textiles selected from natural and synthetic, woven and non-woven.

2. The new composition acts faster than other compositions in obtaining a two log 10 reduction in colony forming units (CFU) of pathogenic organisms, for example E coli and Candida albicans, wherein said other compositions do not contain one of the components of the new composition or contain an alternative component instead of one of the components of the new composition.

3. The new composition is colorless, and does not impart brownish color typical to copper- and silver oxide to textiles to which it is applied.

4. Other masterbatch formulations that are impregnated in various fibers usually have a self-limiting amount of active metal oxides, such as, but not limited to, cuprous oxide, that can be added to the fibers. In the case of very thin fibers, such as those in filament yarns where one denier per filament is common, normally no more than 1% (w/w) can be placed in a filament fiber. It was surprisingly found that using the formulation described herein, the fiber accepted 5% (w/w) of the particulate composition with no production issues and even 10% (w/w) with only a relatively small slowing of production (about 15%). In staple fibers where 3% is usually the upper limit of a metal oxide impregnated in the polymer, there was no problem in arriving at a 10% (w/w) load of the particulate components of the new composition. The obtained fibers are therefore highly potent in various antimicrobial and cosmetic applications, also being exceptionally durable, due to the enhanced concentration of the active metal oxide ingredients.

The term “antimicrobial”, as used herein, refers to an inhibiting, microcidal or oligodynamic effect against microbes, pathogens, and microorganisms, including but not limited to enveloped viruses, non-enveloped viruses, gram-positive bacteria, gram-negative bacteria, fungi, parasites, mold, yeasts, spores, algae, protozoa, acarii and dust mites, amongst others, and subsequent anti-odor properties.

The term “colorless”, as used herein, such as in reference to the antimicrobial composition, means an essentially white composition which does not have specific absorption in the visible region of the solar spectra. In this connection, the terms “colorless” and “white” can be used interchangeably.

As used herein, the term “polymer” refers to materials consisting of repeated building blocks called monomers. The polymer may be homogenous or heterogeneous in its form; hydrophilic or hydrophobic; natural, synthetic, mixed synthetic or bioplastic.

The present invention provides a new composition with antimicrobial properties for impregnation of filaments and staple fibers. Additionally, this composition is white, allowing subsequent dyeing of the treated fibers or filaments without encountering the problem of non-uniform color. The composition comprises: titanium dioxide (TiO₂); a salt comprising silver and phosphate ions, copper oxide; and a mixed oxidation state silver oxide. This composition, and essentially similar compositions, will be denoted herein as “white copper”.

The copper oxide in the composition may be selected from cuprous oxide or cupric oxide or mixtures thereof. Typically, cuprous oxide is preferred.

The term “mixed oxidation state silver oxide”, as used herein, refers to a single silver oxide compound, which contains silver in at least two different oxidation states. In some embodiments, the mixed oxidation state silver oxide contains silver in its I and III oxidation states. In some exemplary embodiments, the mixed oxidation state silver oxide is selected from the group consisting of Ag₄O₄, Ag₂O₂, and mixtures thereof. It has been surprisingly found by the inventors that as little as 0.1% (w/w) of the mixed oxidation silver oxide in the composition was sufficient to achieve the desired antibacterial efficiency of the composition and of the fibers having incorporated therein said composition. However, when the mixed oxidation state silver oxide was not present in the composition, its antimicrobial efficiency was significantly lower.

Titanium dioxide or titanium(IV) oxide (TiO₂) is a well-known color additive in paint, food, drug and cosmetic applications, which is typically used when a white pigment is required. Titanium dioxide has been shown to have antimicrobial activity with potential bactericidal and fungicidal applications in food contact and packaging surfaces. In addition, TiO₂ has other characteristics such as stability, no toxicity, capability of repeated use without substantial loss of catalytic ability, and low cost.

Titanium dioxide is approved by the US Food and Drug Administration for use in food industry (Yemmireddy and Hung, 2015).

In order to mask the reddish color of copper oxide and mixed oxidation state silver oxide in the previously known antimicrobial compositions, relatively large amounts of titanium oxide were required. It has been surprisingly found that despite the antimicrobial activity of TiO₂, the overall antimicrobial activity of the composition has been significantly decreased. Accordingly, inclusion of an additional ingredient was required in order to increase the efficiency of the composition in combating various microbes, viruses, and fungi.

It has been unexpectedly found that rather than an additional metal oxide, a silver phosphate salt provided the highest antimicrobial efficiency, when added to a composition comprising copper oxide, the mixed oxidation state oxide, and TiO₂. Preferably, the salt comprising silver and phosphate ions is an inorganic salt. In some embodiments, said inorganic salt is silver phosphate (Ag₃PO₄). Ag₃PO₄ may be encapsulated by a glass, zirconium or zeolite encapsulant. In some embodiments, said inorganic salt is a zirconium phosphate-based ceramic ion-exchange resin containing silver. In some related embodiments, said inorganic salt is silver sodium hydrogen zirconium phosphate (Ag_{(0.1-0.5)}Na_{(0.1-0.8)}H_{(0.1-0.8)}Zr₂(PO₄)₃). In some embodiments, silver sodium hydrogen zirconium phosphate is selected from the group consisting of Ag_{0.18}Na_{0.57}H_{0.25}Zr₂(PO₄)₃, Ag_{0.46} Na_{0.29}H_{0.25}Zr₂(PO₄)₃, and mixtures thereof. The presence of the salt comprising silver and phosphate ions in the composition was shown to be essential for providing the required antibacterial efficiency of the composition and of the fibers having incorporated therein said composition.

TiO₂ can be present in the composition in a weight percent ranging from about 70 to about 85% of the total weight of the composition. In some embodiments, TiO₂ is present in the composition in a weight percent ranging from about 75 to about 85% of the total weight of the composition. In some exemplary embodiments, TiO₂ is present in the composition in a weight percent of about 80% of the total weight of the composition.

The salt comprising silver and phosphate ions can be present in the composition in a weight percent ranging from about 10 to about 25% of the total weight of the composition. In some embodiments, the salt comprising silver and phosphate ions is present in the composition in a weight percent ranging from about 13 to about 22% of the total weight of the composition. In further embodiments, the salt comprising silver and phosphate ions is present in the composition in a weight percent ranging from about 15 to about 20% of the total weight of the composition. In additional embodiments, the salt comprising silver and phosphate ions is present in the composition in a weight percent ranging from about 10 to about 20% of the total weight of the composition. In other embodiments, the salt comprising silver and phosphate ions is present in the composition in a weight percent ranging from about 15 to about 25% of the total weight of the composition.

In some embodiments, Ag₃PO₄ is present in the composition in a weight percent ranging from about 10 to about 25% of the total weight of the composition. In additional embodiments, Ag₃PO₄ is present in the composition in a weight percent ranging from about 10 to about 20% of the total weight of the composition. In certain embodiments, Ag₃PO₄ is present in the composition in a weight percent of about 15% of the total weight of the composition.

In some embodiments, silver sodium hydrogen zirconium phosphate is present in the composition in a weight percent ranging from about 10 to about 25% of the total weight of the composition. In additional embodiments, silver sodium hydrogen zirconium phosphate is present in the composition in a weight percent ranging from about 15 to about 25% of the total weight of the composition. In certain embodiments, silver sodium hydrogen zirconium phosphate is present in the composition in a weight percent of about 20% of the total weight of the composition.

Copper oxide can be present in the composition in a weight percent ranging from about 0.2 to about 10% of the total weight of the composition. In some embodiments, copper oxide is present in the composition in a weight percent ranging from about 0.2 to about 5% of the total weight of the composition. In further embodiments, copper oxide is present in the composition in a weight percent ranging from about 0.5 to about 2% of the total weight of the composition. In some exemplary embodiments, copper oxide is present in the composition in a weight percent of about 1% of the total weight of the composition.

The mixed oxidation state silver oxide can be present in the composition in a weight percent ranging from about 0.01 to about 1.5% of the total weight of the composition. In some embodiments, the mixed oxidation state silver oxide is present in the composition in a weight percent ranging from about 0.05 to about 0.5% of the total weight of the composition.

According to some embodiments, the weight percentage of the individual components to the total weight of the white copper composition are as follows: TiO₂ 70-85%; the salt comprising silver and phosphate ions 10-25%; copper oxide 0.2-10%; and mixed oxidation state silver oxide 0.01-1.5%. In some embodiments, the weight percentage of the individual components to the total weight of the white copper composition are as follows: TiO₂ 75-85%; the salt comprising silver and phosphate ions 15-25%; copper oxide 1.5-5%; and mixed oxidation state silver oxide 0.01-1.5%. In additional embodiments, the weight percentage of the individual components to the total weight of the white copper composition are as follows: TiO₂ 75-85%; the salt comprising silver and phosphate ions 10-20%; copper oxide 0.5-5%; and mixed oxidation state silver oxide 0.01-1.5%.

In some embodiments, the composition further contains elemental silver. In other embodiments, the above composition further includes elemental zinc. In yet other embodiments, the composition contains both elemental silver and elemental zinc. In still other embodiments the composition contains ZnO alone. In another embodiment the composition includes ZnO and elemental Ag. In some embodiments the composition includes ZnO and elemental Zn and elemental Ag. In some related embodiments, the composition comprises 1.5-5% (w/w) of Zn species out of the total weight of the composition. In further related embodiments, the composition comprises 0.05-0.5% (w/w) of elemental Ag out of the total weight of the composition.

According to some embodiments, the weight percentage of the individual components to the total weight of the white copper composition are as follows: TiO₂ 70-85%; the salt comprising silver and phosphate ions 10-25%; copper oxide 0.2-10%; mixed oxidation state silver oxide 0.01-1.5%; and Zn species 1.5-5%. According to some embodiments, the weight percentage of the individual components to the total weight of the white copper composition are as follows: TiO₂ 70-85%; the salt comprising silver and phosphate ions 10-25%; copper oxide 0.2-10%; mixed oxidation state silver oxide 0.01-1.5%; and Ag 0.05-1.5%. According to some embodiments, the weight percentage of the individual components to the total weight of the white copper composition are as follows: TiO₂ 70-85%; the salt comprising silver and phosphate ions 10-25%; copper oxide 0.2-10%; mixed oxidation state silver oxide 0.01-1.5%; Zn species 1.5-5%; and Ag 0.05-1.5%.

According to one particular embodiment, the weight percentage of the individual components to the total weight of the white copper composition are as follows: TiO₂75%; the salt comprising silver and phosphate ions 16%; copper oxide 4%; mixed oxidation state silver oxide 1%; and Zn species 4%.

The composition can further include an optical brightener. In some embodiments, the optical brightener is present in the composition in the weight percent ranging from about 0.1% to about 2% of the total weight of the composition. The optical brightener can be selected, inter alia, from oxazole, biphenyl, cournarin, stilbene, pyrazolene, rhodamine, fluorescein, and combinations thereof.

Further provided is a masterbatch formulation which includes the above white copper composition. In some embodiments, the white copper composition constitutes about 1-40% of the total masterbatch weight.

Preferably, the masterbatch further comprises a carrier polymer.

The term “carrier polymer”, as used herein, refers to a largest component of a masterbatch formulation which is usually compatible with a substrate polymer.

In some embodiments, the carrier polymer is present in the masterbatch formulation in a weight percent of about 60-99% out of the total weight of the masterbatch formulation. In further embodiments, the carrier polymer is present in the masterbatch formulation in a weight percent of about 70-90% out of the total weight of the masterbatch formulation.

The carrier polymer can be selected from polyethylene, polypropylene, polyester, polybutylene terephthalate (PBT), polyolefins, acrylonitrile-butadiene-styrene (ABS), polyaramids, such as, e.g., nylon 6 or nylon 66, polyurethane, acrylic, polylactic acid, and mixtures thereof, or any polymer used in extrusion molding.

In addition to the following components: TiO₂; the salt comprising silver and phosphate ions; copper oxide; a mixed oxidation state silver oxide, and, optionally, Zn species; and Ag; and the carrier polymer, the masterbatch formulation can further include a wax for encapsulating the white copper composition. The wax can be present in the masterbatch formulation in a percent weight of about 0.1-1% of the total weight of the masterbatch formulation. The wax may be selected from a group of waxes consisting of polyethylene terephthalate (PET), polyester and polyalkene waxes.

The masterbatch formulation may also include a dispersing polymer for dispersing the composition, for example polymethylmethacrylate (PMMA) or silica, at a percent weight of about 0.1-1% of the total weight of the masterbatch formulation. The term “dispersing polymer”, as used herein, refers to a minor component in a masterbatch formulation that allows the other components of the formulation to disperse in a carrier polymer, the latter usually the largest component of the masterbatch formulation.

In some embodiments the masterbatch formulation includes an optical whitening agent. The optical whitening agent can have a percent weight of the total masterbatch formulation of 0.1-1%.

According to some embodiments, the weight percentage of the individual components to the total weight of the masterbatch formulation are as follows: TiO₂ 70-85%; the salt comprising silver and phosphate ions 10-25%; copper oxide 0.2-10%; mixed oxidation state silver oxide 0.01-1.5%; wax 0.1-1%; dispersing polymer 0.1-1%; and, optionally, Zn species 1.5-5%; and Ag 0.05-0.5%.

Typically, the solid components of the composition and of the masterbatch are in a form of a powder (i.e., particulate form). Generally, the components of the composition are 10 micron or less in diameter when used in the masterbatch formulation but larger than 100-150 nanometers. Smaller particle sizes have been found to be very important in optimizing the antimicrobial effect of the composition.

Similarly, it has been found that when the components are more or less the same small size, for example about 0.5 to 2 microns, the antimicrobial effect of the composition is greater.

In some embodiments, the particulates of the components of the composition have a diameter with a D50 ranging from about 100 nm to about 10 μm. In further embodiments, the particulates of the components of the composition have a diameter with a D50 ranging from about 100 nm to about 5 μm. In some embodiments, the particulates of the components of the composition have a diameter with a D90 ranging from about 100 nm and about 10 μm. In further embodiments, the particulates of the components of the composition have a diameter with a D90 ranging from about 100 nm and about 5 μm. In still further embodiments, the particulates of the components of the composition have a diameter with a D50 ranging from about 500 nm and about 2 μm. In yet further embodiments, the particulates of the components of the composition have a diameter with a D90 ranging from about 500 nm and about 2 μm.

The present invention further provides a method for producing polymer filaments. This method comprises the steps of:
providing and melting a substrate polymer resin by passing it through a heated extruder;
adding and melting a masterbatch formulation having the composition discussed above to the melted substrate polymer resin in the extruder; and
extruding a filament containing the masterbatch formulation uniformly distributed in the substrate polymer.

The term “substrate polymer”, as used herein, refers to a polymer into which the masterbatch is placed and to which the properties of the components of the masterbatch are transferred. The substrate polymer can also be called, or thought of, as the “product polymer”.

Non-limiting examples of suitable substrate polymers include polyamide, polyester, acrylic, isotactic compounds including but not limited to polypropylene, polyethylene, polyolefin, acrylic compounds, polyalkene, silicones, and nitrile; cellulose-based polymer or a mixture of different cellulose materials; converted cellulose mixed with plasticizers such as but not limited to rayon viscose, starch-based polymer, and acetate; petroleum derivatives and petroleum gels; fats, both synthetic and natural; polyurethane; natural latex; and mixtures and combinations thereof.

The carrier polymer allows uniform dispersal of the content of the masterbatch in the substrate polymer. In masterbatch compounding, a 20% weight/weight load in a carrier polymer is an unexpectedly large load not often seen. The usual weight/weight ratio of masterbatch to substrate polymer resin is one percent in filament yarns and three percent in staple fibers.

The method above may also contain a step of cutting the filaments into staple fibers.

In some embodiments, the method comprises grinding the colorless antimicrobial composition prior to forming the masterbatch formulation. Preferably, the particulates of the ground composition have a diameter with a D50 ranging from about 100 nm and about 5 μm, more preferably with a D90 ranging from about 100 nm and about 5 μm.

Further disclosed is a method for producing antimicrobial polymer filaments. The method comprises the steps of: providing a melted substrate polymer resin by passing the substrate polymer resin through a heated extruder; and sprinkling the composition discussed above on the external surface of the heated substrate polymer filaments after they emerge from the extruder, thereby imparting antimicrobial properties to the filaments.

The method for producing antimicrobial polymer filaments can further include the step of applying a binder to the filaments prior to sprinkling the white copper composition. In some embodiments, the step of sprinkling comprises spraying.

In some embodiments, the method comprises grinding the colorless antimicrobial composition prior to the step of sprinkling said composition.

Preferably, the particulates of the ground composition have a diameter with a D50 ranging from about 100 nm and about 5 μm, more preferably with a D90 ranging from about 100 nm and about 5 μm.

Further provided is a method for producing antimicrobial natural sliver fibers comprising the steps of: (a) providing at least one ribbon of sliver fibers; (b) dispensing a paste comprising the colorless composition with antimicrobial properties according to the various embodiments presented hereinabove, water and a thickening agent, on the at least one sliver fiber ribbon; and (c) conveying the paste-coated at least one sliver fiber ribbon through a sonotrode.

In some embodiments, the sliver is a cotton sliver.

The sonotrode can be operated between about 500 W to about 3000 W and between about 15 kHz to about 30 kHz.

Additional details on the methods for producing impregnated natural sliver fibers can be found in WO 2019/229756, which content is incorporated herein by reference in its entirety.

In some embodiments, the method comprises grinding the colorless antimicrobial composition prior to forming the paste of step (b). Preferably, the particulates of the ground composition have a diameter with a D50 ranging from about 100 nm and about 5 μm, more preferably with a D90 ranging from about 100 nm and about 5 μm.

Further provided is a material comprising filaments, sliver fibers or staple fibers having incorporated therein the colorless composition with antimicrobial properties according to the various embodiments presented hereinabove.

In some embodiments, the components of the composition are dispersed substantially uniformly throughout the bulk of the filaments, sliver fibers or staple fibers.

As used herein, the term “uniformly” denotes that the volume percentage of the white copper composition particles along the longitudinal axis of the filaments or fibers varies by less than 20%, preferably less than 10%.

The components of the composition may be present in filaments, sliver fibers or staple fibers in a weight percent of about 3-10% out of the total weight of the filament, sliver fiber or staple fiber. In some embodiments, the filaments, sliver fibers or staple fibers comprise at least about 3% (w/w) of the components of the colorless composition. In further embodiments, the filaments, sliver fibers or staple fibers comprise at least about 5% (w/w) of the components of the colorless composition. In certain embodiments, the filaments, sliver fibers or staple fibers comprise at least about 10% (w/w) of the components of the colorless composition.

In some embodiments, at least 0.25% of the total weight of the components of the composition are present on the surface of the filaments, sliver fibers or staple fibers. In further embodiments, at least 0.5% (w/w) of the total weight of the components of the composition are present on the surface of the filaments, sliver fibers or staple fibers. In certain embodiments, about 1% of the total weight of the components of the composition are present on the surface of the filaments, sliver fibers or staple fibers.

The filaments, sliver fibers or staple fibers of the material can be formed into a yarn, a fabric, or a finished textile product.

In some embodiments, said filaments or staple fibers are made from a polymer. In some embodiments, the polymer is the substrate polymer. In some embodiments, the polymer is selected from the group consisting of polyamide, polyester, polyalkene, polysiloxane, nitrile, polyvinyl acetate, starch-based polymer, cellulose, cellulose-based polymer, and mixtures thereof. In some embodiments, the composition is encapsulated in a wax before being incorporated into the polymer. The wax can be selected from the group consisting of PET, polyester, polyalkene waxes, and mixtures thereof.

In some embodiments, said filaments, sliver fibers or staple fibers are made from a natural material. The natural material can be selected from cotton, silk, wool, and mixtures thereof. In some currently preferred embodiments, said natural material is cotton.

A material is also taught having wound healing properties. In some embodiments, the material comprises fibers of a substrate polymer having incorporated therein the composition of white copper as discussed above. In some embodiments, the composition is encapsulated within a wax. In further embodiments, the wax is selected from a group consisting of polyethylene terephthalate (PET), polyester and polyalkene waxes. In stull further embodiments, the substrate polymer is selected from a group consisting of polyamide, polyester, polyalkene, polysiloxane, nitrile, polyvinyl acetate, starch-based polymer, cellulose, cellulose-based polymer, and mixtures thereof. The material having wound healing properties, comprising a substrate polymer having incorporated therein the white copper composition discussed above may be further processed to form yarns and fabrics. The composition incorporated in the substrate polymer may be provided as a masterbatch.

According to some currently preferred embodiments, said material is not characterized by the brownish color of copper- or silver oxide.

The present invention provides for a material having a beneficial cosmetic effect. In some embodiments, the material reduces facial wrinkles, crow's feet, and facial and neck lines, improves skin hydration, reduces mottled hyper-pigmentation, and improves the overall appearance of the skin wherein the material contains a substrate polymer incorporating the white copper composition discussed above. This material can be configured for direct contact with the face and neck requiring cosmetic treatment, allowing components of the white copper composition to be in contact with a fluid. The polymer, herein denoted as “substrate polymer”, can be formed into filaments, staple and sheaths from which yarn and fabrics may be made which then may be formed into facial masks, eye masks, scarfs or other materials for any parts of the body requiring cosmetic treatment. The composition incorporated in the substrate polymer may be provided as a masterbatch.

In some embodiments, the material is for use in combating or inhibiting the activity of microbes or micro-organisms, selected from the group consisting of gram-positive bacteria, gram-negative bacteria, fungi, parasites, mold, spores, yeasts, protozoa, algae, acarii and viruses.

In another aspect, there is provided a colorless composition with antimicrobial properties for impregnation of filaments, sliver fibers and staple fibers, the composition being prepared by mixing the following components: TiO₂; a salt comprising silver and phosphate ions; copper oxide; and a mixed oxidation state silver oxide.

According to some embodiments, the composition is prepared by mixing the following components in the following weight percent to the total weight of the composition: about 70-85% (w/w) TiO₂; about 10-25% (w/w) of the salt comprising silver and phosphate ions; about 0.2-10% (w/w) copper oxide; and about 0.01-1.5% (w/w) of the mixed oxidation state silver oxide.

The composition can further be prepared by mixing at least one of a zinc species and elemental silver (Ag) in addition to the previously mentioned components. The zinc species can be selected from the group consisting of elemental zinc, ZnO, and mixtures thereof. According to some embodiments, Zn species are used at a weight percent of about 1.5-5% (w/w) of the total weight of the composition. According to some embodiments, elemental Ag is used at a weight percent of about 0.05-0.5% (w/w) of the total weight of the composition.

In another aspect, there is provided a method for preparing the colorless composition according to the various embodiments presented hereinabove, the method comprising mixing the following components: TiO₂; a salt comprising silver and phosphate ions; copper oxide; and a mixed oxidation state silver oxide.

According to some embodiments, the method comprises mixing the following components in the following weight percent to the total weight of the composition: about 70-85% (w/w) TiO₂; about 10-25% (w/w) of the salt comprising silver and phosphate ions; about 0.2-10% (w/w) copper oxide; and about 0.01-1.5% (w/w) of the mixed oxidation state silver oxide.

In some embodiments, the method further comprises mixing at least one of a zinc species and elemental silver (Ag) with the previously mentioned components. The zinc species can be selected from the group consisting of elemental zinc, ZnO, and mixtures thereof. According to some embodiments, Zn species are used at a weight percent of about 1.5-5% (w/w) of the total weight of the composition. According to some embodiments, elemental Ag is used at a weight percent of about 0.05-0.5% (w/w) of the total weight of the composition.

The following examples are presented for illustrative purposes only and are to be construed as non-limitative to the scope of the invention.

### EXAMPLES

### Example 1: White Copper Composition Preparation

The following materials were used for the preparation of the white copper composition:

TiO₂— The D50 particle size was 0.5 micron and the powder was purchased from The Cary Company.

Two different silver phosphate compounds were used: (a) Silver Phosphate Zirconimum salt—The D50 was 1 micron and the powder was Alphasan 5000 from Milliken; and (b) Irgaguard® B 7000 by BASF, which is inorganic silver glass, with a particle size of about 2 microns.

Zinc Oxide—The powder size was 0.5 micron and it was purchased from Microban ZO7 or from Wester Reserve CR 1314.

Cu₂O—The powder size was 1.5 microns and it was purchased from Chemet.

The powder has been further ground down to 0.5 microns.

Tetrasilver tetroxide—The as-prepared powder size was 1.5 microns and it was ground down to 0.5 microns. Tetrasilver tetroxide powder was prepared through a reduction process from a silver nitrate solution by a standard procedure known to a person skilled in the art, and as described by Hammer and Kleinberg in Inorganic Synthesis (volume IV, page 12). The basic tetrasilver tetroxide synthesis as referenced above was prepared by addition of NaOH into distilled water, followed by addition of a potassium persulfate and then the addition of silver nitrate.

All the powders were mixed and went through a final grinding process to ensure as even a particle size as possible of all compounds. The weight percentages of the above ingredients within the white copper composition were varied as disclosed in the following examples. The optimal ranges of the ingredients were found to be: TiO₂ 70-85%; the salt comprising silver and phosphate ions 10-25%; copper oxide 0.2-10%; mixed oxidation state silver oxide 0.01-1.5%; and Zn species 1.5-5%.

### Example 2: Incorporation of the White Copper Composition into Polymer Fibers

The mixed powder obtained in Example 1 was added to a high shear mixer which has a hot air blower. Polymethyl methacrylate (PMMA) was added while mixing and allowed to blend for 5 minutes. Wax, such as polyester wax or polyethylene wax, was then added while mixing after the PMMA allowed to blend for 5 minutes.

The mixed treated powder was then placed in a twin-screw master batch machine. The carrier polymer was introduced to the master batch machine. The chemistry was dosed in pellets with a concentration of up to 40% which is a standard industry concentration. The materials were blended in the twin screw mixer which is hot enough to melt the carrier polymer. Each polymer has its own melting temperature and the machine is adjusted accordingly. Master batch pellets were formed.

The master batch was added to the slurry of the extrusion. The weight percentage of the mixed powder in the slurry was dependent on the shape and thickness and the carrier polymer being used. The concentrations used were as follows: filament polyester fibers 3-6% (w/w), filament polypropylene fibers 5-7% (w/w), staple polyester fibers 4-5% (w/w), molded polypropylene 10% (w/w).

### Example 3: Incorporation of the White Copper Composition into Cotton Slivers

The mixed powder obtained in Example 1 was added to a high shear mixer to assure the powder is homogenous. The mixed powder was then added to water with a surfactant and allowed to saturate the cotton which is traveling on a conveyor belt. The individual slivers, being completely saturated with the compound, were then put through the sonication reactor. The cotton was then rinsed to remove extraneous powder and dried.

The treated cotton was then introduced into the yarn spinning process.

### Example 4: Antimicrobial Activity of the White Copper Composition Incorporated into Polymeric Fibers

Antimicrobial activity of polypropylene (PP) fibers prepared as described in Example 2 and comprising the following white copper composition was tested (the numbers refer to weight percentages of the white copper composition components):

TiO₂/Irgaguard B7000/CuO/Ag₄O₄=80/18.9/1/0.1

The composition and the fibers were white in color. The antimicrobial activity was compared to that of copper oxide alone (wherein copper oxide is also incorporated into PP fibers) and to a negative control (PP fibers without any type of antibacterial treatment).

The test was performed as follows:

Following test method AATCC Test Method 100-2017: Two fabrics were prepared for the test. One comprised the white copper composition. The second fabric was a control and was the same as the treated fabric but without the white copper composition.

A finite amount of a sterile serum which contains a known amount of the targeted pathogen or bacteria or virus was placed on both fabrics. Each fabric was then placed in an incubator for a specific amount of time (to be determined by the test desired). The two fabrics were removed and each was allowed to soak in its own beaker of sterile serum. The two fabrics were then removed from their beaker and a sample of the serum was placed on a Petri dish. The two Petri dishes were then put in an incubator and after 48 hours the colonies of bacteria were counted in each.

In all cases 1 Gram+ and 1 Gram− were chosen for the test with common bacteria of E. Coli (Gram−) and Staph. Aureus (Gram−) were used.

The results of the experiment are presented in Table 1 as Activity (CFU/h). It was shown that the white copper composition provided similar antimicrobial activity to that of 100% copper oxide.

TABLE 1
Antimicrobial (Gram− E. coli) activity (CFU/hr)
of PP fibers: 1 - not impregnated, 2 - impregnated with
copper oxide, and 3 - impregnated with white copper.

| Sample | T = 0 | T = 3 hr. | T = 5 hr. | T = 24 hr. |
|---|---|---|---|---|
| negative | 4,330,000 | 22,875,000 | 117,887,500 | 820,625,000 |
| control | | | | |
| 100% copper | 6,208 | 504 | 170 | 174 |
| oxide | | | | |
| white | 3,821 | 504 | 170 | 174 |
| copper | | | | |

### Example 5: Antimicrobial Activity of the White Copper Composition Incorporated into Cotton Slivers

Antimicrobial activity of cotton slivers prepared as described in Example 3 and comprising the following white copper composition was tested (the numbers refer to weight percentages of the white copper composition components):

TiO₂/Irgaguard B7000/CuO/Ag₄O₄=80/18.9/1/0.1

The composition and the slivers were in white in color. The antimicrobial activity was compared to that of copper oxide alone (wherein copper oxide is also incorporated into PP fibers) and to a negative control (PP fibers without any type of antibacterial treatment).

The test was performed as described in Example 4 using Gram+S. aureus.

The results of the experiment are presented in Table 2 as Activity (CFU/h).

A significant difference in antimicrobial activity in cotton was observed between copper oxide alone and white copper. The white copper impregnated fibers demonstrated a stronger antimicrobial effect than copper oxide alone after 24 hours. It can therefore be concluded that the white copper composition not only allows to mask the natural metal oxides color but also provides an increased antimicrobial efficiency, in particular, when applied to cotton.

TABLE 2
Antimicrobial (Gram+ S aureus.) activity (CFU/hr)
of cotton slivers: 1 - not impregnated, 2 - impregnated
with copper oxide, and 3 - impregnated with white copper.

| | Copper | | | | |
|---|---|---|---|---|---|
| | content | | | | |
| Sample | (ppm) | 0 | 3 hr. | 5 hr. | 24 hr. |
| | | Normal. | Normal. | Normal. | Normal. |
| negative control | — | 2,639 | 36,375 | 142,500 | 252,500 |
| copper oxide | 4884 | 1,513 | 160 | 301 | 192 |
| white copper | 87 | 986 | 42 | 2 | 0 |

### Example 6: Effect of the Composition Components on the Antimicrobial Activity in Polymeric Fibers

In order to assess the significance of each one of the components of the white copper composition for its antimicrobial efficiency, as well as for preserving its white color in polymeric (PP) fibers, a set of experiments was performed, wherein some of the components were missing from the mixture. The effect of the components' concentration was also evaluated. The tested compositions are summarized in Table 3. Silver phosphate used in this experiment was Irgaguard B7000.

TABLE 3
Test compositions (concentrations are provided in
% (w/w) of the total weigh of the composition)

| | formulation | | | | Color | |
|---|---|---|---|---|---|---|
| | | Silver | | | of the | Extrusion |
| Sample* | TiO₂ | phosphate | CuO | Ag₄O₄ | mixture | in PP |
| No. 1 | | | 100.0% | | Brown | 3.0% |
| No. 2 | 80.0% | 19.0% | 1.0% | | White | 2.0% |
| No. 3 | 80.0% | 18.9% | 1.0% | 0.1% | | 2.0% |
| No. 4 | 89.0% | 9.9% | 1.0% | 0.1% | | 2.0% |
| No. 5 | 93.9% | 5.0% | 1.0% | 0.1% | | 2.0% |

The test was performed as described in Example 4.

The results of the experiment are presented in Table 4 as Activity (CFU/h).

TABLE 4
Antimicrobial activity (CFU/hr) of PP fiber samples summarized in Table 3.

| | | | | | % |
|---|---|---|---|---|---|
| | | | | | reduction |
| | | | | | after |
| Sample | 0 | 3 hr. | 5 hr. | 24 hr. | 24 hr. |
| Negative | 4,330,000 | 22,875,000 | 117,887,500 | 820,625,000 | |
| control | | | | | |
| No. 1 | 6,208 | 504 | 170 | 174 | 97.20 |
| No. 2 | 47,350 | 9,513 | 7,950 | 13,588 | 71.30 |
| No. 3 | 3,821 | 504 | 170 | 174 | 95.44 |
| No. 4 | 5,329 | 978 | 1,768 | 975 | 81.70 |
| No. 5 | 7,051 | 4,580 | 4,321 | 3,269 | 53.64 |

It can be seen that the white copper composition comprising all the components as according to the principles of the present invention (i.e., copper oxide, a mixed oxidation state silver oxide, a salt comprising a silver and phosphate ion, and titanium oxide) provided essentially the same antimicrobial efficiency in polymeric fibers as copper oxide (Samples Nos. 1 and 3), while being white in color. It was also shown that the presence of Ag₄O₄ is essential for obtaining the desired antimicrobial efficiency, which is similar to that of copper oxide (Samples Nos. 2 and 3). An effect of the weight percent of the silver phosphate-based component was also shown—its increase (at the expense of titanium oxide contents) enhanced the antimicrobial activity of the composition (Samples Nos. 3, 4, and 5).

### Example 7: Effect of the Composition Components on the Antimicrobial Activity in Cotton Slivers

In order to assess the significance of each one of the components of the white copper composition for its antimicrobial efficiency, as well as for preserving its write color in natural (cotton) fibers, a set of experiments was performed, wherein some of the components were missing from the mixture. The tested compositions are summarized in Table 5.

TABLE 5
Test compositions (concentrations are provided in % (w/w) of the
total weight of the composition)

| | formulation | | | | Copper | Color of |
|---|---|---|---|---|---|---|
| | | Silver | | | content | the |
| Sample | TiO₂ | phosphate | CuO | Ag₄O₄ | (ppm) | mixture |
| No. 6 | | | 91.0% | 9.0% | 8650 | Brown |
| No. 7 | 80.0% | | 20.0% | | 1888 | Pink- |
| No. 8 | 80.0% | | 19.0% | 1.0% | 2543 | Light |
| | | | | | | brown |
| No. 9 | 80.0% | 20%* | | | NA | White |
| No. 10 | 80.0% | 19.9%* | | 0.1% | NA | White |
| No. 11 | 80.0% | 19.9%** | | 0.1% | NA | White |
| No. 12 | | | 100.0% | | 10936 | Brown |
| No. 13 | | | 100.0% | | 1607 | Off white |
| (bleached) | | | | | | |
| No. 14 | 80.0% | 18.9%*** | 1.0% | 0.1% | 150 | white |
| No. 15 | 80.0% | 18.9%*** | 1.0% | 0.1% | 150 | white |

*silver phosphate ground to 2.8 micron
**RC5000 (silver sodium hydrogen zirconium phosphate), particle size 1 micron
***Irgaguard B7000, particle size around 2 microns

Antimicrobial efficiency of the various compositions has been tested against bacteria (Klebsiella pneumoniae) and yeast/mold (Candida Albicans). The test was performed as described in Example 4.

The results of the experiment are presented in Tables 6 and 7 as Activity (CFU/h).

TABLE 6
Antimicrobial (Klebsiella pneumoniae) activity (CFU/hr)
of cotton sliver samples summarized in Table 5.

| | | | | | % reduction |
|---|---|---|---|---|---|
| Sample | 0 | 5 hr. | 24 hr. | 48 hr. | after 24 hr. |
| Negative | 3190 | 29638 | 168750 | 236750 | |
| control | | | | | |
| No. 6 | 948 | 23 | 14 | 7 | 98.48 |
| No. 7 | 2,178 | 4,394 | 4,258 | 3,390 | −95.45 |
| No. 8 | 2,282 | 2,655 | 3,288 | 3,084 | −44.07 |
| No. 9 | 1,858 | 787 | 1,509 | 770 | 18.79 |
| No. 10 | 1,977 | 164 | 87 | 78 | 95.6 |
| No. 11 | 1,839 | 484 | 80 | 58 | 95.6 |
| No. 12 | 1,012 | 20 | 16 | 8 | 98.4 |
| No. 13 | 2,222 | 3,107 | 3,283 | 2,635 | −47.7 |
| Negative | 5,785 | 462,250 | 462,250 | | |
| control | | | | | |
| No. 14 | 3,775 | 36 | 8 | | 99.79 |

TABLE 7
Antimicrobial (Candida Albicans) activity (CFU/hr)
of cotton sliver samples summarized in Table 5.

| | | | | | % reduction |
|---|---|---|---|---|---|
| | 0 | 5 hr. | 24 hr. | 48 hr. | after 24 hr. |
| Negative | 192 | 1557 | 7670 | 107614 | |
| control | | | | | |
| No. 6 | 60 | 10 | 3 | 3 | 94 |
| No. 7 | 141 | 250 | 1935 | 15409 | −1270 |
| No. 8 | 144 | 218 | 1494 | 14017 | −937 |
| No. 9 | 122 | 85 | 686 | 3500 | −463 |
| No. 10 | 128 | 15 | 33 | 182 | 74 |
| No. 11 | 117 | 43 | 19 | 188 | 84 |
| No. 12 | 70 | 9 | 8 | 4 | 89 |
| No. 13 | 147 | 193 | 1457 | 12625 | −891 |
| Negative | 194,988 | 203,238 | 2,880,000 | 3,748,000 | |
| control | | | | | |
| No. 15 | 138,988 | 22,625 | 9,713 | 20,000 | 93 |

As mentioned hereinabove, the previously known antimicrobial combination of copper oxide and Ag₄O₄ had an intense brown color, as well as copper oxide alone (Sample Nos. 6 and 12). In order to minimize color intensity in the cotton fibers, they were bleached, which lead to the reduction in their microbial efficiency against yeast/mold (Sample No. 12).

In order to provide a colorless antibacterial formulation, the white copper composition was prepared, including a combination of copper oxide, a mixed oxidation state silver oxide, a salt comprising a silver and phosphate ion, and titanium oxide. It can be seen that as in polymeric fibers, cotton impregnated with the white copper composition provided essentially the same antimicrobial efficiency against bacteria and yeast/mold as copper oxide (Samples Nos. 12, 14 and 15), while being white in color.

It was also shown that the presence of Ag₄O₄ and the silver-phosphate-based component is essential for obtaining the desired antimicrobial efficiency, as well as for providing a colorless composition (Samples Nos. 7, 14 and 15). Addition of Ag₄O₄ without the silver phosphate-based component was not sufficient for increasing the antimicrobial efficiency of the composition and masking the reddish color (Samples Nos. 8, 14, and 15). Similarly, a combination of titanium oxide and the silver phosphate-based component, which was used without Ag₄O₄ and copper oxide has a relatively low antibacterial activity (Samples Nos. 9, 14 and 15). While addition of Ag₄O₄ to the combination of titanium oxide and the silver phosphate-based component increased the antimicrobial activity of the composition, its efficiency was still lower than that of the white copper composition, in particular against yeast/mold (Samples Nos. 10, 11, 14, and 15). Different sources of the silver-phosphate based compound did not significantly affect the efficiency of the composition (Sample Nos. 10 and 11).

### Example 8: Effect of the Particle Size of the Composition Components on the Antimicrobial Activity in Cotton Slivers

In order to evaluate the impact of particle size on antibacterial activity, different formulations of white copper were tested to compare Irgaguard B7000 vs silver phosphate (having different particle sizes) and copper oxide vs nano-copper oxide

Nano-copper oxide (40-80 ppm) was tested instead of copper oxide used in Examples 1-3 hereinabove, to determine the impact of nanoparticles on the antibacterial activity. While copper oxide is typically used at 1% (w/w) concentration, nano-copper oxide was tested at two different concentrations: 0.5% (w/w) and 1% (w/w).

Silver phosphate is the main component of Irgaguard B7000 mixed with 18% of zinc and 0.5% silver. Silver phosphate was tested alone instead of Irgaguard B7000 to evaluate the effect of the particle size of this component compared to Irgaguard B7000. The particle size of Irgaguard B7000 is between 2 and 2.5 microns, while that of silver phosphate is around 4 microns.

Antimicrobial efficiency of the various compositions has been tested against bacteria (E-Coli). The test was performed as described in Example 4.

The different formulations and the anti-bacterial efficiency results of the study are presented in Table 8.

TABLE 8
Antimicrobial (E. coli) activity (CFU/hr) of cotton slivers impregnated with
white copper compositions with different particle sizes of the components

| Sample | formulation | 0 | 3 hr. | 5 hr. | 24 hr. |
|---|---|---|---|---|---|
| Neg. Control | — | 2,716 | 29,113 | 223,250 | 181,000 |
| No. 16 | TiO₂/Irgaguard/CuO/ | 1,153 | 84 | 8 | 2 |
| | Ag₄O₄ = 80/18.9/1/0.1 | | | | |
| No. 17 | TiO₂/Irgaguard/Nano | 1,521 | 418 | 17 | 12 |
| | CuO/Ag₄O₄ = | | | | |
| | 80/18.9/1/0.1 | | | | |
| No. 18 | TiO₂/Silver Phosphate/ | 1,287 | 151 | 96 | 98 |
| | CuO/Ag₄O₄ = | | | | |
| | 80/18.9/1/0.1 | | | | |
| No. 19 | TiO₂/Silver Phosphate/ | 1,388 | 1,270 | 235 | 1,765 |
| | nanoCuO/Ag₄O₄ = | | | | |
| | 80/18.9/1/0.1 | | | | |

It can be seen that nano-copper oxide is less efficient than micron-sized copper oxide. Both with Irgaguard B7000 and silver phosphate, nano-copper oxide provided inferior efficiency as compared to micron-sized copper oxide. Without wishing to being bound by theory or mechanism of action, it is contemplated that material properties can change when the particle size is decreased below 100 nm. Accordingly, the preferable particle size of copper oxide is about 1 micron.

Silver phosphate was found to be less efficient than Irgaguard B7000 with the same concentration of 18.9% in the final formulation and micron-sized copper oxide, which could be due to its larger particle size. In order to assess the effect of the particle size of silver phosphate, the white copper composition comprising silver phosphate was ground to reduce the mean particle size of its components, and in particular that of silver phosphate. The ground composition had the following particle size parameters: D50=5.98; D90=44.1; and 63% of the particles had a particle size below 10 μm.

The antimicrobial efficiency of the ground composition incorporated into copper slivers is shown in Table 9.

TABLE 9
Antimicrobial (E. coli) activity (CFU/hr) of cotton slivers
impregnated with ground white copper composition.

| Sample | description | 0 | 5 hr. | 24 hr. | 48 hr. |
|---|---|---|---|---|---|
| Negative | | 3850 | 38475 | 366250 | 366250 |
| Control | | | | | |
| No. 20 | TiO₂/Irgaguard/CuO/Ag₄O₄ = 80/18.9/1/0.1 | 2047 | 168 | 27 | 8 |
| No. 21 | TiO₂/SP/CuO/Ag₄O₄ = 80/18.9/1/0.1 | 611 | 77 | 99 | 99 |
| No. 22 | TiO₂/SP/CuO/Ag₄O₄ = 80/18.9/1/0.1 | 603 | 53 | 12 | 5 |

It can be seen that the mean particle size of the white copper composition particulate components correlates with the antimicrobial activity. Reducing the particle sizes of the composition components such that D50 is about 5 μm, improves the antibacterial efficiency of the composition, such that a silver phosphate salt can be used without any additives or encapsulants.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1-44. (canceled)

45. A colorless composition with antimicrobial properties for impregnation of filaments, sliver fibers and staple fibers, the composition comprising:
titanium dioxide (TiO₂);
a salt comprising silver and phosphate ions;
copper oxide; and
a mixed oxidation state silver oxide.

46. The composition according to claim 45, wherein copper oxide is selected from the group consisting of cuprous oxide, cupric oxide, and mixtures thereof, and wherein the salt comprising silver and phosphate ions is selected from the group consisting of silver phosphate (Ag₃PO₄), silver sodium hydrogen zirconium phosphate (Ag_{(0.1-0.5)}Na_{(0.1-0.8)}H_{(0.1-0.8)}Zr₂(PO₄)₃), and mixtures thereof.

47. The composition according to claim 46, wherein silver sodium hydrogen zirconium phosphate is selected from the group consisting of Ag_{0.18}Na_{0.57}H_{0.25}Zr₂(PO₄)₃, Ag_{0.46}Na_{0.29}H_{0.25}Zr₂(PO₄)₃, and mixtures thereof, and wherein Ag₃PO₄ is encapsulated by a glass, zirconium, or zeolite encapsulant.

48. The composition according to claim 45, wherein the mixed oxidation state silver oxide is selected from the group consisting of Ag₄O₄, Ag₂O₂, and mixtures thereof.

49. The composition according to claim 45, further comprising at least one of a zinc species or elemental silver (Ag), wherein the zinc species is selected from the group consisting of elemental zinc, ZnO, and mixtures thereof.

50. The composition according to claim 45, wherein the composition exhibits the following components in the following weight percentages out of a total weight of the composition:
wherein the TiO₂ is present in an amount of about 70-85% (w/w);
wherein the salt comprising silver and phosphate ions is present in an amount of about 10-25% (w/w); wherein the copper oxide is present in an amount of about 0.2-10% (w/w); and
wherein the mixed oxidation state silver oxide is present in an amount of about 0.01-1.5% (w/w).

51. The composition according to claim 50, further comprising about 1.5-5% (w/w) Zn species and/or 0.05-0.5% (w/w) elemental Ag out of the total weight of the composition.

52. The composition according to claim 45, wherein particulates of the components of the composition have a diameter with a D50 ranging from about 100 nm and about 5 μm.

53. A masterbatch formulation, comprising:
the composition according to claim 45; and
a carrier polymer;
wherein the carrier polymer is present in the masterbatch formulation in a weight percent of about 60-99% out of the total weight of the masterbatch formulation and is selected from the group of polymers consisting of polyethylene, polypropylene, polybutylene terephthalate (PBT), polyolefins, acrylonitrile-butadiene-styrene (ABS), polyaramids, and mixtures thereof.

54. The masterbatch formulation according to claim 53, further comprising a wax for encapsulating the components of the composition, wherein the wax is present in the masterbatch formulation in a weight percent of about 0.1-1% out of the total weight of the masterbatch formulation, and is selected from the group consisting of polyethylene terephthalate (PET), polyester, polyalkene waxes and mixtures thereof.

55. The masterbatch formulation according to claim 53, further comprising a dispersing polymer for dispersing the components of the masterbatch formulation in the carrier polymer, wherein the dispersing polymer is present in the masterbatch formulation in a weight percent of about of 0.1-1.0% out of the total weight of the masterbatch formulation and is selected from the group consisting of polymethylmethacrylate (PMMA) and silica.

56. A method for producing antimicrobial polymer filaments, the method comprising:
providing and melting a substrate polymer by passing the substrate polymer through a heated extruder;
adding the masterbatch formulation of claim 53 to the melted substrate polymer; and
extruding a filament containing the masterbatch formulation uniformly dispersed therein, wherein the masterbatch formulation constitutes about 1-10% (w/w) of the substrate polymer.
wherein the substrate polymer is selected from the group consisting of polyethylene, polypropylene, polybutylene terephthalate, polyolefins, ABS, polyaramids, and mixtures thereof.

57. The method of claim 56, further comprising cutting the filament into staple fibers.

58. A material, comprising:
filaments, sliver fibers, or staple fibers having incorporated therein the composition according claim 45;
wherein the components of the composition are dispersed substantially uniformly throughout a bulk of the filaments, sliver fibers, or staple fibers; and
wherein at least 0.25% of the total weight of the components of the composition are present on a surface of the filaments, sliver fibers, or staple fibers.

59. The material according to claim 58, wherein said material is formed into a yarn, a fabric, or a finished textile product, wherein said filaments or staple fibers are made from a polymer and wherein said polymer is selected from the group consisting of polyamide, polyester, polyalkene, polysiloxane, nitrile, polyvinyl acetate, starch-based polymer, cellulose, cellulose-based polymer, and mixtures thereof.

60. The material according to claim 59, wherein the composition is encapsulated in a wax before being incorporated into the polymer, wherein the wax is selected from the group consisting of polyethylene terephthalate (PET), polyester, polyalkene waxes, and mixtures thereof.

61. The material according to claim 58, wherein said filaments, sliver fibers or staple fibers are made from a natural material selected from the group consisting of cotton, silk, wool, and mixtures thereof.

62. The material according to claim 58, wherein the material comprising the filaments, sliver fibers or staple fibers is formed into facial masks, eye masks, scarves, clothing items, bedding textiles, medical textiles, bandages or sutures.

63. The material according to claim 58, for use in a cosmetic treatment, selected from the group consisting of reducing wrinkles, reducing crows-feet, reducing skin hyper-pigmentation, reducing facial and neck lines, reducing erythema, reducing edema, softening of skin, and improving skin elasticity, wherein the filaments, sliver fibers or staple fibers are in direct contact with part of a user's face or neck requiring said cosmetic treatment.

64. The material according to claim 58, for use in skin regeneration processes, selected from the group consisting of wound healing, accelerated wound closure, and wound healing with reduced scarring.
